(19) United States
(12) Patent Application Publication
(10) Pub. No.: US 2023/0010377 A1
(43) Pub. Date: Jan. 12, 2023

(54) DEVICE AND METHOD FOR PROVIDING FODDER INFORMATION OPTIMIZED FOR INDIVIDUALS

(71) Applicant: SEOUL NATIONAL UNIVERSITY R&DB FOUNDATION, Seoul (KR)

(72) Inventor: Seong Chan YEON, Seoul (KR)

(21) Appl. No.: 17/784,618

(22) PCT Filed: Dec. 21, 2020
(86) PCT No.: PCT/KR2020/018736

§ 371 Date: Jun. 10, 2022

(30) Foreign Application Priority Data
Dec. 20, 2019 (KR) 10-2019-0171652
Dec. 18, 2020 (KR) 10-2020-0178224

(51) Int. Cl. G06N 5/04; A23K 10/16; A23K 50/00

(52) U.S. Cl. CPC G06N 5/04; A23K 10/16; A23K 50/00

(57) ABSTRACT

A food information providing device optimized for each individual according to an embodiment of the present disclosure includes a data acquisition unit configured to acquire characteristic information and microbiome analysis result information of a target individual, and a processor configured to determine a food customized for the target individual based on the acquired characteristic information and microbiome analysis result information.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2020/018736, filed Dec. 21, 2020, which claims priority to the benefit of Korean Patent Application Nos. 10-2019-0171652 filed on Dec. 20, 2019 and 10-2020-0178224 filed on Dec. 18, 2020, in the Korean Intellectual Property Office, the entire contents of which are incorporated herein by reference.

### BACKGROUND

### 1. Technical Field

The present disclosure relates to a device and method for providing food information optimized for each individual.

### 2. Background Art

Recently, as the number of pet households has rapidly increased, the need for a food recommendation service for pets is being strongly raised. As the number of types of food has rapidly increased along with an increase in the number of pet households, pet owner are always sensitive to food feeding. However, since it is difficult for the pet owners to determine whether the corresponding food is suitable for pets, it is common to purchase food based on brand awareness only.

However, the pets vary in types and breeds, and the nutrients that the pet should consume vary depending on its age, size, physical condition, disease, etc. Therefore, it is not easy for the pet owners to choose food that contains the necessary nutrients customized for their pets.

Accordingly, a research on technology that recommends food optimized for each pet is continuing.

### SUMMARY

The present disclosure provides a device and method for providing food information optimized for each individual.

In one aspect of the present disclosure, there is provided a food information providing device optimized for each individual comprising a data acquisition unit configured to acquire characteristic information and microbiome analysis result information of a target individual, and a processor configured to determine a food customized for the target individual based on the acquired characteristic information and microbiome analysis result information.

The characteristic information may include a type, a breed, a weight, an age, a sex, an obesity status, a disease status, a blood test result, and a neutering surgery of an individual.

The processor may be configured to determine nutrients required for the target individual based on the acquired characteristic information and microbiome analysis result information and determine a food composition capable of providing the determined nutrients.

The food composition may include a type, a quality, and a quantity of components of the food.

The processor may be configured to determine a health state of the target individual based on the acquired characteristic information and microbiome analysis result information, and when the determined health state is abnormal, determine types and a ratio of harmful bacteria and beneficial bacteria in the target individual using the microbiome analysis result information, determine types and an amount of beneficial bacteria required for the target individual, and determine the food composition considering the types and the amount of beneficial bacteria required for the target individual.

The food information providing device may further comprise an output unit configured to output information on the determined food.

The food information may include a food type, product information, and a dietary method.

The processor may be configured to use a food recommendation model trained to determine an optimal food customized for an individual based on characteristic information and microbiome analysis result information.

In another aspect of the present disclosure, there is provided a method of providing food information optimized for each individual, the method comprising acquiring characteristic information and microbiome analysis result information of a target individual, and determining a food customized for the target individual based on the acquired characteristic information and microbiome analysis result information.

The characteristic information may include a type, a breed, a weight, an age, a sex, an obesity status, a disease status, a blood test result, and a neutering operation of an individual.

Determining the food customized for the target individual may comprise determining nutrients required for the target individual based on the acquired characteristic information and microbiome analysis result information, and determining a food composition capable of providing the determined nutrients.

The food composition may include a type, a quality, and a quantity of components of the food.

Determining the food composition may comprise determining a health state of the target individual based on the acquired characteristic information and microbiome analysis result information, and when the determined health state is abnormal, determining types and a ratio of harmful bacteria and beneficial bacteria in the target individual using the microbiome analysis result information, determining types and an amount of beneficial bacteria required for the target individual, and determining the food composition considering the types and the amount of beneficial bacteria required for the target individual.

The method may further comprise outputting information on the determined food.

The food information may include a food type, product information, and a dietary method.

Determining the food customized for the target individual may comprise determining the food customized for the target individual using a food recommendation model trained to determine an optimal food customized for an individual based on characteristic information and microbiome analysis result information.

The present disclosure can recommend food optimized for each individual based on characteristics and a microbiome analysis result of individuals. Hence, a user can easily select food of individual.

### BRIEF DESCRIPTION OF THE DRAWINGS

FIG. 1 illustrates a food information providing device optimized for each individual according to an exemplary embodiment.

FIG. 2 illustrates a food information providing device optimized for each individual according to an exemplary embodiment.

FIG. 3 illustrates a method of providing food information optimized for each individual according to an exemplary embodiment.

### DETAILED DESCRIPTION

Reference will now be made in detail to embodiments of the disclosure, examples of which are illustrated in the accompanying drawings. Wherever possible, the same reference numbers will be used throughout the drawings to refer to the same or like parts. It will be noted that a detailed description of known arts will be omitted if it is determined that the detailed description of the known arts can obscure the embodiments of the disclosure.

For respective steps, respective steps may be performed in a different order from the stated order unless the context clearly indicates a specific order. That is, the respective steps may be performed in the same order as specified, or may be performed substantially simultaneously, or may be performed in the reverse order.

The terms to be described later are terms defined in consideration of functions in the present disclosure and may vary depending on intentions or customs, etc. of users or operators. Therefore, the definition should be made based on the contents throughout the present disclosure.

The terms including an ordinal number such as first, second, etc. may be used to describe various components, but the components are not limited by such terms. The terms are used only for the purpose of distinguishing one component from other components. A singular expression can include a plural expression as long as it does not have an apparently different meaning in context. In the present disclosure, terms “include” and “have” should be understood to be intended to designate that illustrated features, numbers, steps, operations, components, parts or combinations thereof are present and not to preclude the existence of one or more different features, numbers, steps, operations, components, parts or combinations thereof, or the possibility of the addition thereof

In the present disclosure, the classification of components is merely classified based on the main function for which each component is responsible. That is, two or more components may be combined into one component, or one component may be divided into two or more components for each more subdivided function. Each of the components may additionally perform all or part of function of another component in addition to the main function for which each component is responsible, and part of the main function of each component may be exclusively performed by another component. Each component may be implemented as hardware or software, or may be implemented as a combination of hardware and software.

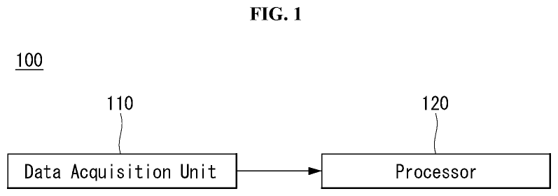

FIG. 1 illustrates a food information providing device optimized for each individual according to an exemplary embodiment. A food information providing device 100 optimized for each individual (hereinafter, referred to as a ‘food information providing device’) illustrated in FIG. 1 is a device for providing food information optimized for a target individual based on characteristics and a microbiome analysis result of the target individual, and may be mounted on an electronic device or may be surrounded by a housing and formed as a separate device. Examples of the electronic device may include a personal computer, a mobile phone, a smartphone, a tablet, a laptop, a personal digital assistant (PDA), a portable multimedia player (PMP), a navigation device, an MP3 player, a digital camera, a wearable device, and the like. Examples of the wearable device may include a glasses type, a wrist watch type, a wristband type, a ring type, an earring type, a belt type, a necklace type, an ankle band type, a thigh band type, a forearm band type, and the like. The electronic device is not limited to the examples described above, and the wearable device is not limited to the examples described above.

Referring to FIG. 1, the food information providing device 100 according to an exemplary embodiment may include a data acquisition unit 110 and a processor 120.

The data acquisition unit 110 may acquire characteristic information of the target individual and information of microbiome analysis result of the target individual. Herein, the individual may include human and animal that consume food (e.g., feed, fodder, etc.), and the characteristic information of the individual may include a type (e.g., human, dog, cat, etc.), breed, weight, age, sex, an obesity status, a disease status, a blood test result, a neutering surgery of individual, and the like.

For example, the data acquisition unit 110 may acquire the characteristic information of the target individual from a hospital (including a veterinary hospital) server that measures and/or stores characteristic information of individuals, and acquire the microbiome analysis result of the target individual from a company server that performs microbiome analysis of individuals. In this instance, the data acquisition unit 110 may use wired and wireless communication technology. Herein, the wireless communication technology may include Bluetooth communication, Bluetooth Low Energy (BLE) communication, near field communication (NFC), WLAN communication, Zigbee communication, Infrared Data Association (IrDA) communication, Wi-Fi direct (WFD) communication, ultra-wideband (UWB) communication, Ant+ communication, Wi-Fi communication, radio frequency identification (RFID) communication, 3G communication, 4G communication, 5G communication, and the like, but is not limited thereto.

As another example, the data acquisition unit 110 may receive the characteristic information and the microbiome analysis result information of the target individual from the user through a predetermined input means to acquire the characteristic information of the target individual and the microbiome analysis result information of the target individual.

The processor 120 may control an overall operation of the food information providing device 100.

The processor 120 may determine an optimal food for the target individual based on the characteristic information and the microbiome analysis result information of the target individual, and output information on the determined food through an output means. The food information may include food type, product information, dietary methods, etc., and the product information may include product name, manufacturer, food components, nutrients, additives, etc.

According to an embodiment, the processor 120 may determine an optimal food composition customized for the target individual based on the characteristic information and the microbiome analysis result information of the target individual. The food composition may include type, quality, quantity, etc. of components of food.

Nutrients required for each individual may vary depending on the characteristics of each individual, such as the type, breed, weight, age, sex, obesity status, disease status, blood test result, neutering surgery, etc. of each individual and/or the microorganisms present in each individual. Accordingly, according to an embodiment, the processor 120 may determine nutrients required for the target individual based on the acquired characteristic information and the microbiome analysis result information of the target individual, and may determine an optimal food composition that can provide the determined nutrients, for example, type, quality, quantity, etc. of components of food. In this instance, the processor 120 may consider components of food that cannot be consumed for each type of individuals.

For example, the processor 120 may determine whether a health state of the target individual is normal or abnormal based on the characteristic information and the microbiome analysis result information of the target individual. If it is determined that the health state of the target individual is abnormal, the processor 120 may determine types and a ratio of harmful bacteria and beneficial bacteria in the target individual using the microbiome analysis result information, and determine types and an amount of beneficial bacteria required for the target individual. In this instance, the processor 120 may consider type and ratio of beneficial bacteria that exist in an individual with a normal health state. Further, the processor 120 may determine an optimal food composition customized for the target individual considering the types and amount of beneficial bacteria required for the target individual.

The processor 120 may determine a microbiome product including beneficial bacteria required for the target individual and output information of the determined microbiome product through an output means. The microbiome product information may include a product name, a manufacturer, and types and an amount of microorganisms, and the like. Multiple microbiome product information may be previously stored in a database inside or outside the food information providing device 100, and the processor 120 may search the database to determine a microbiome product customized for the target individual.

When the optimal food composition customized for the target individual is determined, the processor 120 may determine the optimal food customized for the target individual based on the determined food composition. In this instance, the processor 120 may consider quality and quantity of food ingredients that help food components. Multiple food information may be previously stored in a database inside or outside the food information providing device 100, and the processor 120 may search the database to determine optimal food for the target individual.

According to an embodiment, the processor 120 may determine the optimal food for the target individual using a food recommendation model.

The food recommendation model may be a machine learning model trained to determine an optimal food customized for individual based on characteristic information and microbiome analysis result information of individuals. For example, the food recommendation model may be previously generated through machine learning based on learning data including characteristics and microbiome analysis result of each individual for various individuals, and food corresponding to them. The machine learning model may include artificial neural network, decision tree, genetic algorithm, genetic programming, K-nearest neighbor, radial basis function network, random forest, support vector machine, and deep-learning, and the like.

The processor 120 may periodically update the food recommendation model. For example, the processor 120 may continue to collect additional learning data, for example, actual data on the target individual (characteristics and microbiome analysis result of the target individual and the corresponding optimal food), and periodically additionally learn it using the collected additional learning data to update the food recommendation model. A performance of the food recommendation model can be improved by continuously updating the food recommendation model. That is, as the number of usages of the food recommendation model increases, it can learn with more data. Therefore, the performance of the food recommendation model can be further improved by an increase in the number of usages.

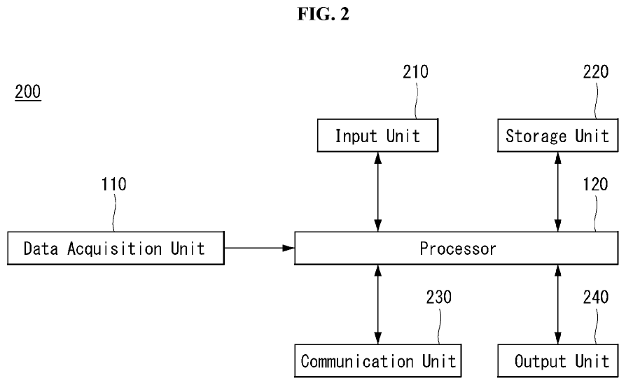

FIG. 2 illustrates a food information providing device optimized for each individual according to an exemplary embodiment.

Referring to FIG. 2, a food information providing device 200 may include a data acquisition unit 110, a processor 120, an input unit 210, a storage unit 220, a communication unit 230, and an output unit 240. Since the data acquisition unit 110 and the processor 120 are the same as those described above with reference to FIG. 1, a detailed description thereof is omitted.

The input unit 210 may receive various operating signals and information from a user. According to an embodiment, the input unit 210 may include a key pad, a dome switch, a touch pad, a jog wheel, a jog switch, an H/W button, and the like. In particular, when the touch pad forms an inter-layer structure with a display, it may be referred to as a touch screen.

The storage unit 220 may store programs or commands for an operation of the food information providing device 200, and store input data and processed data of the food information providing device 200, data required to determine food optimized for a target individual, and the like.

The storage unit 220 may include at least one type of storage medium such as, a flash memory type, a hard disk type, a multimedia card micro type, a card type memory (e.g., SD or XD memory, etc.), a random access memory (RAM), a static random access memory (SRAM), a read only memory (ROM), an electrically erasable programmable read only memory (EEPROM), a programmable read only memory (PROM), a magnetic memory, a magnetic disk, or an optical disk. The food information providing device 200 may operate an external storage medium such as web storage that performs a storage function of the storage unit 220 on the Internet.

The communication unit 230 may communicate with an external device. For example, the communication unit 230 may transmit input data, stored data, processed data, etc. of the food information providing device 200 to the external device, or receive, from the external device, various data required to determine food optimized for the target individual.

The communication unit 230 may communicate with the external device using wired and wireless communication technology. The wireless communication technology may include Bluetooth communication, Bluetooth Low Energy (BLE) communication, near field communication (NFC), WLAN communication, Zigbee communication, Infrared Data Association (IrDA) communication, Wi-Fi direct (WFD) communication, ultra-wideband (UWB) communication, Ant+ communication, Wi-Fi communication, radio frequency identification (RFID) communication, 3G communication, 4G communication, 5G communication, and the like, but is merely an example and is not limited thereto.

The output unit 240 may output input data, stored data, processed data, etc. of the food information providing device 200. According to an embodiment, the output unit 240 may output acquired characteristic information and microbiome analysis result information of the target individual, and optimal food composition information, optimal food information, and microbiome product information determined based on them through at least one method of an auditory method, a visual method, and a tactile method. To the end, the output unit 240 may include a display, a speaker, a vibrator, and the like.

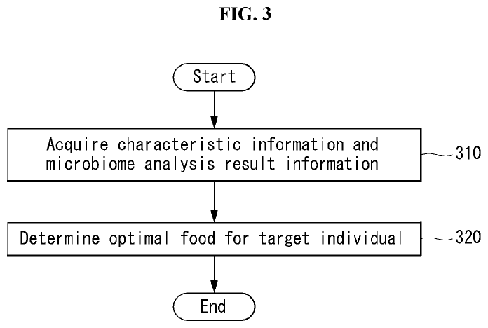

FIG. 3 illustrates a method of providing food information optimized for each individual according to an exemplary embodiment. The method of providing food information illustrated in FIG. 3 may be performed by the food information providing device 100 or 200 of FIG. 1 or 2.

Referring to FIG. 3, a food information providing device may acquire characteristic information of a target individual and microbiome analysis result information of the target individual, in 310.

For example, the food information providing device may acquire the characteristic information of the target individual from a hospital (including a veterinary hospital) server that measures and/or stores characteristic information of individuals, and acquire the microbiome analysis result of the target individual from a company server that performs microbiome analysis of individuals.

As another example, the food information providing device may receive the characteristic information and the microbiome analysis result information of the target individual from a user through a predetermined input means to acquire the characteristic information of the target individual and the microbiome analysis result information of the target individual.

The food information providing device may determine an optimal food for the target individual based on the characteristic information and the microbiome analysis result information of the target individual, and output information on the determined food, in 320. The food information may include food type, product information, dietary methods, etc., and the product information may include product name, manufacturer, food components, nutrients, additives, etc.

According to an embodiment, the food information providing device may determine an optimal food composition customized for the target individual based on the characteristic information and the microbiome analysis result information of the target individual. The food composition may include type, quality, quantity, etc. of components of food. For example, the food information providing device may determine nutrients required for the target individual based on the acquired characteristic information and the microbiome analysis result information of the target individual, and may determine an optimal food composition that can provide the determined nutrients, for example, type, quality, quantity, etc. of components of food. In this instance, the food information providing device may consider components of food that cannot be consumed for each type of individuals.

For example, the food information providing device may determine whether a health state of the target individual is normal or abnormal based on the characteristic information and the microbiome analysis result information of the target individual. If it is determined that the health state of the target individual is abnormal, the food information providing device may determine types and a ratio of harmful bacteria and beneficial bacteria in the individual using the microbiome analysis result information, and determine the types and amount of beneficial bacteria required for the target individual. Further, the food information providing device may determine an optimal food composition customized for the target individual considering the type and ratio of beneficial bacteria required for the target individual.

The food information providing device may determine a microbiome product including beneficial bacteria required for the target individual and output information of the determined microbiome product. The microbiome product information may include a product name, a manufacturer, and types and amount of microorganisms, and the like.

When the optimal food composition customized for the target individual is determined, the food information providing device may determine the optimal food customized for the target individual based on the determined food composition. In this instance, the food information providing device may consider quality and quantity of food ingredients that help food components.

According to another embodiment, the food information providing device may determine the optimal food for the target individual based on the characteristic information and the microbiome analysis result information of the target individual, and a food recommendation model.

The food recommendation model may be a machine learning model trained to determine an optimal food customized for individual based on characteristic information and microbiome analysis result information of individuals. For example, the food recommendation model may be previously generated through machine learning based on learning data including characteristics and microbiome analysis result of each individual for various individuals, and food corresponding to them.

The device and method for providing food information according to an exemplary embodiment may complete an optimal food composition customized for the target individual based on the characteristic information and the microbiome analysis result information of the target individual, determine food close to the completed food composition, and provide information on the food. In this instance, microbiome product information required for the target individual may be provided together, if necessary or desired. The completed food composition may be used to newly product food of the target individual.

The device and method for providing food information according to an exemplary embodiment may be used in a field of animal insurance to set an insurance rate. That is, the food information providing device and method may cause disease decrease/increase, etc. based on food customized for each individual, and may be used to calculate the insurance rate based on this result. For example, animals that are fed based on the food information providing device and method according to an exemplary embodiment may be given a relatively lower insurance rate than other animals.

The embodiments described above may be implemented using a computer-readable medium with programs recorded thereon to perform various methods presented herein. The computer-readable medium may include all kinds of recording devices storing data that is readable by a computer system. Examples of the computer-readable medium may include ROM, RAM, CD-ROM, a magnetic tape, a floppy disk, an optical disc, and the like. The computer-readable medium may be distributed into computer systems connected via networks and may be executed by being written in computer-readable codes through a distribution method.

So far, the present disclosure has been described with reference to a number of illustrative embodiments thereof. It should be understood that numerous other modifications and embodiments can be implemented by those skilled in the technical field to which the present disclosure pertains within the scope of the principles of the present disclosure. Accordingly, the scope of the present disclosure is not limited to embodiments described above, and various variations and modifications are possible in the component parts and/or arrangements of the subject combination arrangement within the scope of the appended claims.

1. A food information providing device optimized for each individual, comprising:
a data acquisition unit configured to acquire characteristic information and microbiome analysis result information of a target individual; and
a processor configured to determine a food customized for the target individual based on the acquired characteristic information and microbiome analysis result information.

2. The food information providing device of claim 1, wherein the characteristic information includes a type, a breed, a weight, an age, a sex, an obesity status, a disease status, a blood test result, and a neutering surgery of an individual.

3. The food information providing device of claim 1, wherein the processor is configured to determine nutrients required for the target individual based on the acquired characteristic information and microbiome analysis result information and determine a food composition capable of providing the determined nutrients.

4. The food information providing device of claim 3, wherein the food composition includes a type, a quality, and a quantity of components of the food.

5. The food information providing device of claim 3, wherein the processor is configured to:
determine a health state of the target individual based on the acquired characteristic information and microbiome analysis result information; and
when the determined health state is abnormal, determine types and a ratio of harmful bacteria and beneficial bacteria in the target individual using the microbiome analysis result information, determine types and an amount of beneficial bacteria required for the target individual, and determine the food composition considering the types and the amount of beneficial bacteria required for the target individual.

6. The food information providing device of claim 1, further comprising:
an output unit configured to output information on the determined food.

7. The food information providing device of claim 6, wherein the food information includes a food type, product information, and a dietary method.

8. The food information providing device of claim 1, wherein the processor is configured to use a food recommendation model trained to determine an optimal food customized for an individual based on characteristic information and microbiome analysis result information.

9. A method of providing food information optimized for each individual, the method comprising:
acquiring characteristic information and microbiome analysis result information of a target individual; and
determining a food customized for the target individual based on the acquired characteristic information and microbiome analysis result information.

10. The method of claim 9, wherein the characteristic information includes a type, a breed, a weight, an age, a sex, an obesity status, a disease status, a blood test result, and a neutering surgery of an individual.

11. The method of claim 9, wherein determining the food customized for the target individual comprises:
determining nutrients required for the target individual based on the acquired characteristic information and microbiome analysis result information; and
determining a food composition capable of providing the determined nutrients.

12. The method of claim 11, wherein the food composition includes a type, a quality, and a quantity of components of the food.

13. The method of claim 11, wherein determining the food composition comprises:
determining a health state of the target individual based on the acquired characteristic information and microbiome analysis result information; and
when the determined health state is abnormal, determining types and a ratio of harmful bacteria and beneficial bacteria in the target individual using the microbiome analysis result information, determining types and an amount of beneficial bacteria required for the target individual, and determining the food composition considering the types and the amount of beneficial bacteria required for the target individual.

14. The method of claim 9, further comprising:
outputting information on the determined food.

15. The method of claim 14, wherein the food information includes a food type, product information, and a dietary method.

16. The method of claim 9, wherein determining the food customized for the target individual comprises:
determining the food customized for the target individual using a food recommendation model trained to determine an optimal food customized for an individual based on characteristic information and microbiome analysis result information.
